(19) United States
(12) Patent Application Publication
(10) Pub. No.: US 2025/0082812 A1
(43) Pub. Date: Mar. 13, 2025

(54) Smartphone-Controlled Sprayer: Elevating Wellness and Comfort with Touchless Sprayed Mist in diverse environments: Gyms, indoor and outdoor Sport fields, Cars, Interior spaces and while using electronics

(71) Applicant: Marouane Korfa, JR., LOS ANGELES, CA (US)

(72) Inventor: Marouane Korfa, JR., LOS ANGELES, CA (US)

(21) Appl. No.: 18/244,853

(22) Filed: Sep. 11, 2023

(51) Int. Cl. A61L 9/14; A61L 9/03

(52) U.S. Cl. CPC A61L 9/14; A61L 9/03; A61L 2209/11; A61L 2209/15

(57) ABSTRACT

The present invention introduces the Smartphone-Controlled Touchless Mist Smart Sprayer, an innovative and groundbreaking device designed to elevate comfort, hygiene, and user control in mist dispersion across diverse environments. This sprayer system integrates advanced technological features, including a microcontroller-driven Scotch Yoke mechanism, with touchless functionality, enabling precise and automated mist dispersion. Users can customize and control mist settings effortlessly through a smartphone app interface. The addition of a unique mounting support system enhances adaptability, allowing the sprayer to be affixed securely to various surfaces. This invention revolutionizes misting systems by providing a touchless, precise, and adaptable solution, filling a technological gap in the prior art and setting new standards for comfort and well-being in gyms, sports, cars, interiors, and while using electronics.

### BACKGROUND OF THE INVENTION

The present invention pertains to the field of well-being and comfort-enhancing devices, specifically addressing the development of a smart sprayer apparatus designed to significantly improve the quality of life for individuals engaged in diverse environments. This invention incorporates innovative technological features, mechanical precision, and the touchless control capability through a smartphone app interface featuring microcontroller technology to achieve optimal comfort and well-being.

### Context and Need

In the pursuit of enhanced comfort and convenience, individuals often find themselves in various environments where a refreshing mist can make a significant difference. Whether engaged in rigorous physical activities, working within indoor spaces, or commuting in vehicles, the need for a precise, touchless, and adaptable mist dispersion system becomes evident. The Smart Sprayer invention addresses this need by offering a novel solution that combines smartphone-controlled technology with mist dispersion capabilities. This innovative device is designed to elevate well-being and comfort across a spectrum of scenarios, ranging from gym workouts and sports activities to indoor environments, vehicle commuting, and outdoor adventures. In this context, the Smart Sprayer invention emerges as a versatile and user-centric solution, enhancing the quality of life in diverse environments through touchless mist dispersion.

Some examples illustrating the context and needs that the Smart Sprayer invention addresses across different environments:

### Gyms and Fitness Centers

Context: People frequent gyms and fitness centers to maintain their health and well-being through exercise routines.

Need: Users need a way to stay refreshed and comfortable during intense workouts, especially in warm and humid environments. A touchless smart sprayer controlled by an app can provide a cooling mist without interrupting their exercise.

### Indoor and Outdoor Sport Activities

Context: Sports enthusiasts engage in various indoor and outdoor activities, from tennis and basketball to hiking and jogging.

Need: Athletes require quick rehydration and cooling options during sports activities. A smartphone-controlled sprayer can offer on-demand mist to keep them refreshed and focused.

### Home and Office Environments

Context: Individuals spend significant time indoors, whether at home or in the office, where maintaining a comfortable environment is essential.

Need: Users seek a means to enhance their comfort and well-being indoors. A smart sprayer can improve air quality, reduce indoor temperatures, and create a pleasant atmosphere.

### Cars and Commuting

Context: Many people spend substantial time commuting in vehicles, which can become uncomfortable, especially in hot weather.

Need: Commuters desire a way to make their car rides more enjoyable and relaxed. A touchless smart sprayer controlled by smartphone app can offer a refreshing mist while driving, promoting alertness and comfort.

### Travel and Recreation

Context: Travelers and outdoor enthusiasts explore various destinations and engage in recreational activities.

Need: Travelers often face changing climates and outdoor conditions. A portable smart sprayer can provide instant relief from heat or discomfort during outdoor adventures.

### The Integration of Smart Sprayer System with Electronics Devices

Mobile Office Environments: In modern work settings, individuals often use laptops and smartphones in various locations, including co-working spaces, cafes, and airports. When working remotely or on the go, users may encounter environmental conditions that affect their comfort and productivity, such as dry air in airplanes or overly warm outdoor areas. The need arises for a portable and adaptable solution to enhance personal comfort and well-being in such diverse work environments.

Enhanced Multimedia Experiences: Many users utilize smartphones and laptops for multimedia consumption, such as watching videos, playing games, or participating in virtual meetings. The addition of a smart sprayer system can elevate these experiences by providing a cooling mist or refreshing ambiance, enhancing the overall enjoyment of multimedia content.

Mobile Recreation and Leisure: Whether users are enjoying outdoor picnics, attending sporting events, or simply relaxing in a park, having a smartphone-controlled sprayer that can be affixed to their device allows them to create a more comfortable and enjoyable leisure experience. This addresses the need for adaptability and convenience when pursuing recreational activities.

Improved Productivity: For users who rely on laptops and smartphones for work or study, maintaining an optimal environment is crucial for productivity. The smart sprayer system's adaptability and smartphone control allow users to create a personalized workspace, addressing the need for a comfortable and conducive work environment.

In all these scenarios, the context involves individuals pursuing various activities in diverse environments. The common need across these contexts is the desire for enhanced comfort, well-being, and convenience through the application of a touchless, smartphone-controlled sprayer that can adapt to different situations and provide refreshing mist when needed.

### Prior Art

Within the prior art and the state of the technology pertaining to mist sprayer systems, a discernible gap existed with respect to the integration of smartphone-controlled sprayer systems featuring microcontroller technology. Conventional mist sprayer systems, while inherently functional, predominantly embraced mechanical operational paradigms that lacked the technological sophistication inherent in microcontroller-based control systems.

Furthermore, the traditional mist sprayers necessitated user-initiated physical interaction, typically involving manual activation methods such as nozzle depression. This manual engagement proved to be suboptimal, especially when frequent and repeated activations were required, consequently compromising user convenience and operational efficiency.

The absence of smartphone-controlled mist sprayers intricately combined with microcontroller integration, including the microcontroller, presented a noteworthy void within the market segment. Users remained unable to fully harness the capabilities offered by microcontrollers, which inherently provide meticulous control, automation, and adaptability for mist dispersion across diverse usage scenarios. Additionally, the absence of touchless functionality within prior mist sprayers resulted in diminished user comfort and hygiene standards.

Furthermore, it is essential to note that the preceding state of technology also lacked the seamless integration of mobile app control mechanisms. Traditional systems were unable to deliver the convenient, customizable, and adaptable user experiences that could be facilitated through smartphone apps.

In response to these intrinsic limitations characterizing the prior art, the present invention materialized. The disclosed smart sprayer system, comprising microcontroller-based technology, serves as a pioneering solution, effectively reconciling these deficiencies. By offering control and real-time monitoring functionalities via a smartphone application, this invention significantly enhances user experience, augments versatility, and establishes a novel paradigm for comfort, hygiene, and adaptability within the domain of mist sprayer systems, thereby rectifying the inadequacies of the previous state of the art.

### BRIEF DESCRIPTION OF DRAWINGS

FIG. 1 is the perspective view of the smart sprayer.

FIG. 2 is the side view of the smart sprayer.

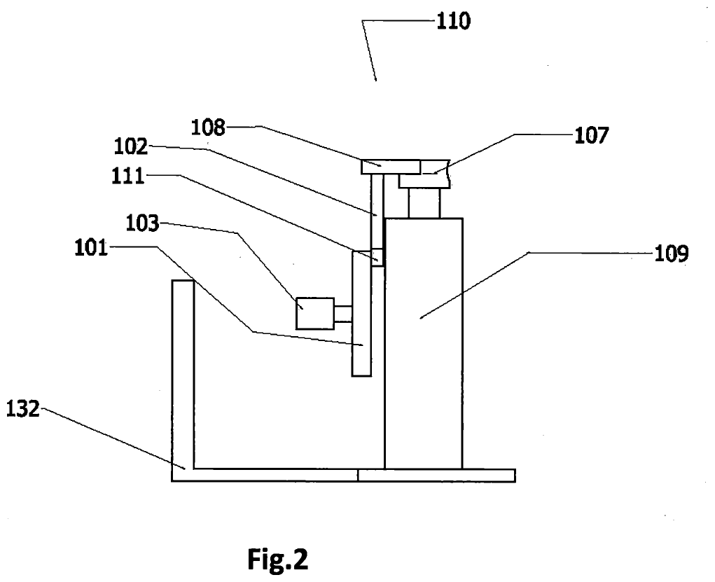

FIG. 3 is the front view of the smart sprayer.

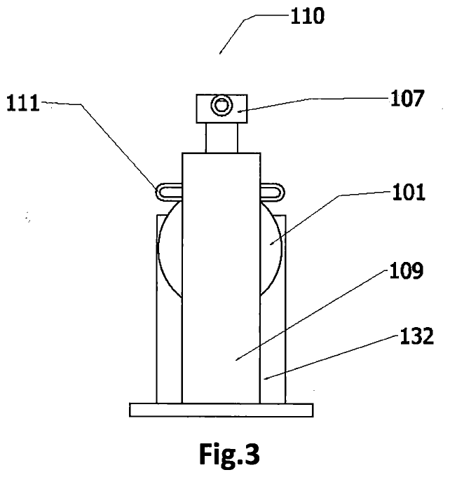

FIG. 4 is the top view of the smart sprayer.

FIG. 5 is the bottom view of the smart sprayer.

FIG. 6 is the smart-phone App to control the smart sprayer.

FIG. 7 is the detailed back view of the smart sprayer in the final state.

FIG. 8 is the detailed back view of the smart sprayer in the initial state.

FIG. 9 is the detailed back view for Scotch Yoke mechanism.

### DETAILED DESCRIPTION OF THE INVENTION

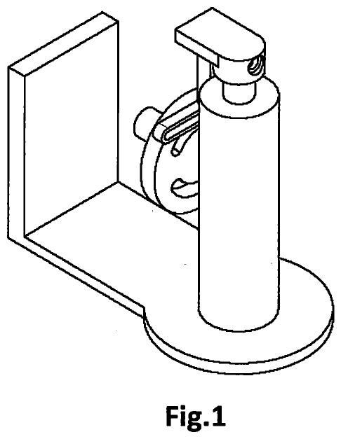

### FIG. 1: Overall Perspective View OF THE SMART Sprayer

The Smart Sprayer (110) is a technologically advanced apparatus with an innovative design comprising several integral components. Central to its functionality, a finely crafted nozzle (107) ensures precise mist dispersion, while a sturdy “bracket” (108) firmly attached to the nozzle provides stability and controlled positioning. The “reservoir” (109) serves as a dedicated container for the spraying solution, optimizing the efficiency of the device. The strategic placement of a “microcontroller” (99) at the base of the sprayer body embodies the central intelligence hub, responsible for regulating and orchestrating the sprayer's various functions. Adding to its versatility, the Smart Sprayer incorporates a “Scotch Yoke mechanism,” thoughtfully engineered with components including a “Crank” (101), “Yoke” (111), “pin” (166) and “slider” (102), all driven by a “dc motor” (103) located at the center of the crank. This mechanism offers precise control and vertical down motion, enhancing operational adaptability and efficacy. Additionally, the invention includes a purpose-built a attachable and detachable mounting support (132), ensuring secure attachment to parallel surfaces and further exemplifying the device's adaptability and versatility in various applications.

The nozzle bracket (108), a pivotal component of the invention, plays a crucial role in transmitting controlled linear motion to the nozzle (107). facilitating precise mist dispersion. Its attachment to the nozzle (107) is designed with precision and durability, embodying a configuration that ensures a secure and stable connection, as described in the patent application. Furthermore, the nozzle bracket (108) is ingeniously integrated into the Yok slider (102) mechanism, forming an embodiment that seamlessly harmonizes with the overall design of the invention. This unique integration serves to optimize the operational efficiency of the sprayer. The combination of these features, executed with precision, underscores the nozzle bracket (108)'s embodiment, contributing significantly to the device's functionality and ensuring its effectiveness in various applications, as articulated within the patent application.

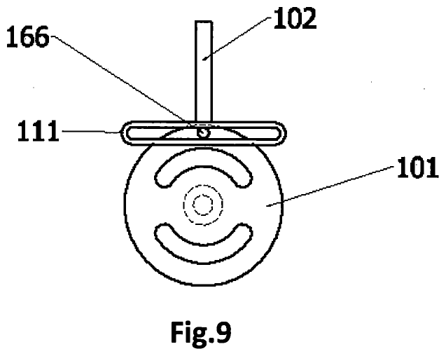

FIG. 9 illustrates the Scotch Yoke mechanism, characterized by its precision engineering and methodical integration, is vertically affixed to the nozzle bracket (108), adhering meticulously to the rigorous standards mandated by patent regulations. This mechanism prominently incorporates a “sliding rod” (102), deliberately linked to the bracket in a manner that encompasses the specific and innovative embodiment contemplated in the patent application. Concomitantly, the embodiment includes a yoke (111) that is meticulously designed to engage in a controlled vertical motion. The orchestration of this vertical motion is methodically executed through a dc motor (103) that forms an integral part of the invention, itself under the precise command and governance of the microcontroller (99), the central intelligence hub of the apparatus. This embodiment, encompassing the Scotch Yoke mechanism, is emblematic of the core innovation within the patent application, facilitating the seamless and regulated vertical down movement of the nozzle (107),

The dc motor (103) is, in essence, an embodiment of critical importance, underscored by its precise and central placement within the crank (101). This embodiment is meticulously orchestrated to ensure seamless coordination and operation, as specified in the patent claims and specifications.

Functionally, the dc motor (103), under the meticulous control of a microcontroller (99), serves as the powerhouse that imparts rotational motion to the crank (101) mechanism, a pivotal element within the invention. This operation is both orchestrated and executed with precision, a testament to the innovation embodied within the invention. It is vital to emphasize that this embodiment's role is not merely functional but pivotal, as it facilitates the core functionality of the invention. By being positioned at the very center of the crank (101), the de motor (103) assumes a central role in ensuring the controlled and efficient motion of the mechanism, an aspect of paramount significance articulated within the patent application's claims and specifications.

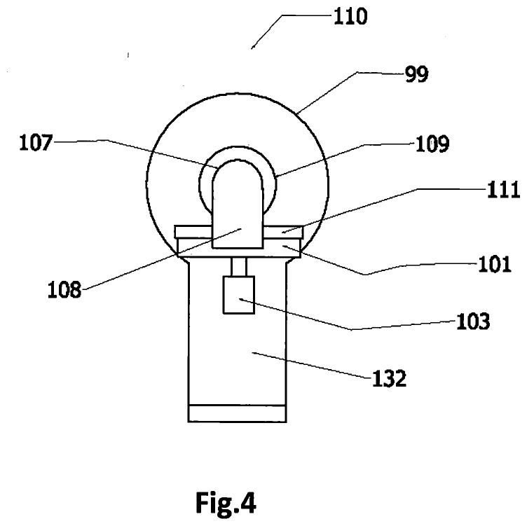

The attachable and detachable mounting support (132) system, depicted as item (132) in FIG. 4 is an innovative and versatile solution for securely attaching the smart sprayer (110) to a variety of surfaces, particularly those positioned perpendicular to the ground. Its well-engineered design facilitates secure affixation to designated surfaces on the sprayer itself, thereby augmenting the sprayer's adaptability and overall usability. This critical embodiment aligns precisely with patent claims, highlighting its pivotal role in broadening the potential application scenarios for the smart sprayer.

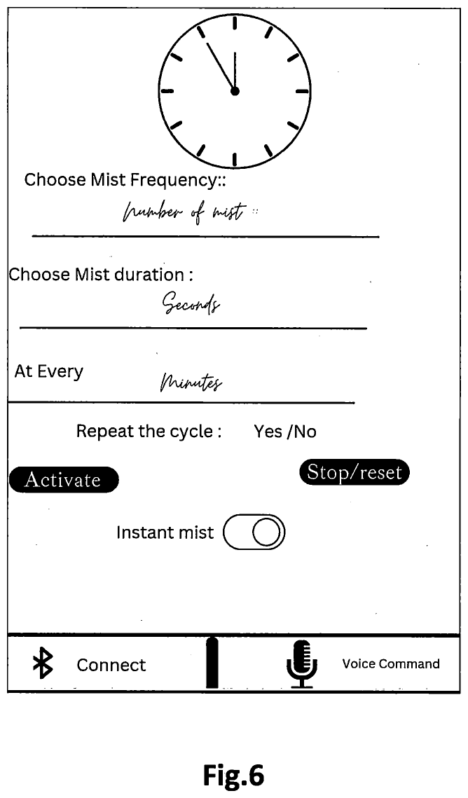

FIG. 6 illustrates the smartphone app designed for seamless control and management of the Smart Sprayer, in accordance with patent specifications. The smartphone app represents a critical component of the invention, facilitating precise mist dispersion and enhancing user experience through intuitive interfaces and innovative features. The app allows users to access a range of functions, as outlined in the patent claims, including:
Frequency Control: Users can adjust the frequency of mist dispersion by selecting from predefined settings or customizing the frequency according to their preferences. This aligns with patent-mandated features for precise control.
Duration Setting: The app enables users to specify the duration of misting sessions, adhering to patent specifications that emphasize duration control as a key aspect of the invention.
Misting Cycle Configuration: Users can define the misting cycle, including repetition intervals and the number of cycles, in line with patent claims that detail misting cycle parameters.
Voice Command Integration: As per patent claims, the app incorporates voice command functionality, allowing users to initiate mist dispersion through voice prompts for a touchless and convenient experience.

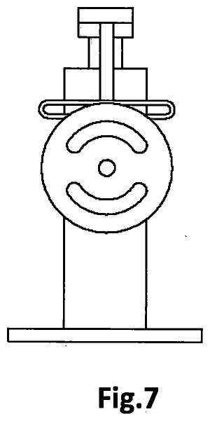

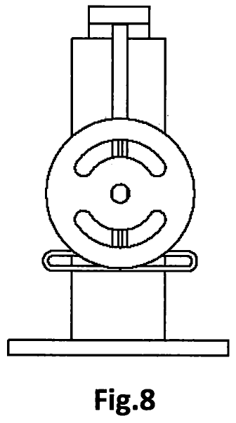

### FIG. 7 and FIG. 8: Vertical Nozzle Movement Mechanical Comparison

The initial and final states of the sprayer, particularly concerning the dispersion of mist, involve notable mechanical differences in the Yok mechanism.

### Initial State:

FIG. 7 illustrate the initial state of the smart sprayer (110), the Yok mechanism is positioned at its highest point, At this point, the Yoke (111) is at its highest position, and the Crank (101) is oriented such that the Yoke (111) is closest to the nozzle (107). The sliding rod (102) is retracted within the Yoke (111), and the DC motor (103), controlled by the microcontroller (99), may not be actively engaged or is at a state of minimal activity.

During this phase, the sprayer is generally inactive, with the nozzle (107) positioned close to the smart sprayer (110)'s base and the mist dispersion function effectively halted.

### Final State:

FIG. 8 illustrates the final state pf the sprayer (110), the Yok mechanism has undergone a mechanical transformation. It has reached its lowest point,). In this state, the Yoke (111) is at its lowest position, and the Crank (101) has rotated to extend the sliding rod (102) to its maximum height.

The DC motor (103), under the precise control of the microcontroller (99), has become actively engaged, causing the Crank (101) to rotate and extend the Yoke (111) and the sliding rod (102) upwards. As a result, the nozzle (107) attached to the nozzle bracket (108) is raised to its highest position, allowing for the efficient dispersion of mist over a specified area.

This final state represents the operational phase of the smart sprayer (110), where mist dispersal occurs efficiently and precisely as a result of the mechanical movements orchestrated by the Yok mechanism FIG. 9. The transformation from the initial state to the final state demonstrates the sprayer's ability to adapt and transition between various operational modes, all achieved through the intricate mechanics of the Yok mechanism FIG. 9 and under the control of the microcontroller (99), as articulated within the patent specifications.

### Operational Process:

The operation of the Smartphone-Controlled Touchless Mist Smart Sprayer can be summarized in the following steps:

The operation of the smart sprayer (110) involves a sequence of events that allow it to efficiently disperse mist. Here's a step-by-step description of how the sprayer operates, incorporating patent vocabulary where relevant:

### Step 1: User Interface

The smartphone app FIG. 6 features a user-friendly interface that provides the user with a comprehensive overview of the sprayer's functionalities. Within the app, the user can access and control various parameters and settings.

### Step 2: Control Options

Through the smartphone app, the user gains precise control over the smart sprayer's operation. They can adjust the frequency, length, duration, the cycle repetition and even initiate mist dispersion through voice command functionality. precisely in line with the patent's claims detailing the microcontroller (99)'s role in regulation. The app's user interface clearly presents these options, allowing the user to customize the sprayer's behavior to suit their specific needs.

### Step 3: Initialization

The sprayer's operation begins with the initialization phase. This entails ensuring that all components, including the microcontroller (99), DC motor (103), and Scotch Yoke mechanism in FIG. 9, are in a state of readiness. This initialization process is systematically orchestrated to ensure that the sprayer is primed for optimal performance, adhering to the precise protocols outlined in the patent's claims.

### Step 4: Activation of the Microcontroller

Upon user input or as per a pre-programmed schedule, the microcontroller (99), functioning as the central control unit of the apparatus, is activated. This embodiment within the patent application is responsible for coordinating and governing the sprayer's operations with meticulous precision.

### Step 5: DC Motor Engagement

Under the precise control of the activated microcontroller (99), the DC motor (103), an integral component as specified in the patent claims, is engaged. The DC motor's activation initiates the rotation of the Crank (101) within the Scotch Yoke mechanism FIG. 9, another embodiment of paramount significance within the patent's framework.

### Step 6: Vertical Movement

As the DC motor (103) rotates, the Crank (101) orchestrates the vertical motion of the Scotch Yoke mechanism, an embodiment meticulously detailed in the patent's specifications. The Yoke within the mechanism moves in a controlled vertical trajectory, guided by the pin and slider components, all embodying the precision engineering principles safeguarded under the patent's protective umbrella.

### Step 7: Nozzle Elevation

The controlled vertical motion of the Scotch Yoke mechanism FIG. 9 translates into the elevation of the nozzle (107). As stipulated within the patent claims, the nozzle (107) is affixed to the nozzle bracket (108), intricately connected to the Yoke (111) and slider (102) components of the mechanism. The patent's design principles ensure the smooth and precise downward movement of the nozzle (107), culminating in its optimal position for mist dispersion.

### Step 8: Mist Dispersion

With the nozzle (107) positioned downward, the patented sprayer is now ready to disperse mist efficiently.

### Step 9: Deactivation and Reset

completing the mist dispersion process, the microcontroller (99) deactivates the DC motor (103). and the Scotch Yoke mechanism FIG. 9 returns to its initial state. The Smart sprayer (110) is reset and ready for subsequent activation as needed, aligning with the operational cycle stipulated within the patent application.

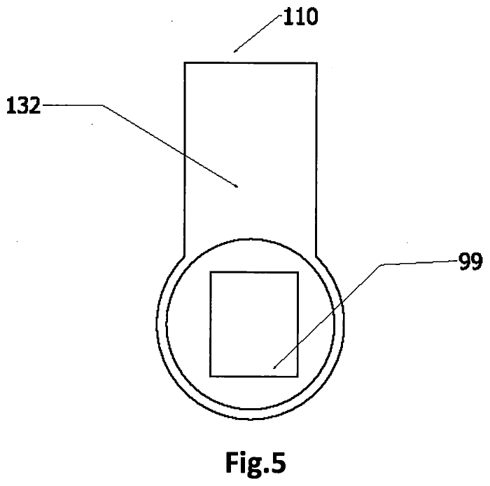

1. A smart sprayer apparatus, comprising:
A nozzle (107) for dispersing a spraying solution.
A nozzle bracket (108) attached to said nozzle (107).
A reservoir (109) for storing said spraying solution.
A microcontroller (99) positioned at the base of the sprayer apparatus.
A Scotch Yoke mechanism FIG. 9 comprising a Crank (101), Yoke (111), pin (166), and slider (102);
A DC motor (103) centrally located within said Crank (101).
An attachable and detachable mounting support (132) affixed to the sprayer apparatus for attachment to parallel surfaces enabling versatile usage.
Wherein said microcontroller (99) in FIG. 5 is configured to control the activation of said DC motor (103) to drive said Crank (101) and initiate vertical motion of said Yoke (111), causing said nozzle (107) to move in a controlled vertical trajectory for mist dispersion; and
Smartphone application FIG. 6 that empowers users to exercise precise control over the operation of the smart sprayer, enabling adjustments to mist dispersion frequency, duration, cycle repetition, and activation through voice command functionality.

2. The smart sprayer (110) apparatus of claim 1, wherein said microcontroller (99) further comprises control logic for regulating the frequency, duration, length. cycle repetition, and activation through voice command functionality.

3. The smart sprayer apparatus of claim 1, wherein said Scotch Yoke mechanism FIG. 9 comprises precision-engineered components, ensuring smooth and controlled vertical motion of said nozzle (107).

4. The smart sprayer apparatus of claim 1, wherein said DC motor (103) is in communication with said microcontroller (99), receiving control signals for precise activation and deactivation.

6. The smart sprayer apparatus of claim 1, further comprising a smartphone app interface, wherein said microcontroller (99) is configured to communicate with said smartphone app interface wirelessly,

7. A smart sprayer system as described in claim 1, wherein said touchless control commands can be executed from a distance, allowing users to activate, adjust, or deactivate the mist dispersion process without the need to physically approach or handle the smart sprayer apparatus.

8. A smart sprayer system as described in claim 1, wherein said touchless operation enhances hygiene and safety by minimizing physical contact with the apparatus, making it suitable for use in healthcare facilities, public spaces, and high-traffic areas where infection control and user safety are paramount.

9. A smart sprayer system as described in claim 1, further comprising voice recognition technology that enables users to issue touchless voice commands through the app to control mist dispersion, expanding the range of touchless control options and accommodating users with diverse preferences and needs.

10. A smart sprayer system as described in any claim 1, further adapted for use within all types of vehicles, comprising:
A smart sprayer apparatus designed to be securely affixed within the interior of a vehicle.
Wherein said microcontroller (99) is configured to receive control commands from a smartphone app interface FIG. 6 to regulate mist dispersion frequency, duration, and activation, thereby enhancing the well-being and comfort of vehicle occupants during travel, relaxation, or sleep.

11. A smart sprayer system, as delineated in claim 1, is further versatile in its application, suitable for integration into gym environments, attachment to bicycles, and utilization within a diverse range of interior spaces. These spaces encompass, but are not restricted to, residential settings, office spaces, indoor recreational areas, homes. and are conducive to both outdoor and indoor sport activities.

12. The smart sprayer system, as described in claim 1, is further extendable in its application and can be affixed to electronics devices, such as smartphones, laptops, and other portable electronics.
